(19) United States
(12) Patent Application Publication
(10) Pub. No.: US 2011/0045520 A1
(43) Pub. Date: Feb. 24, 2011

(54) FATTY ACID MARKERS FOR THE DIAGNOSIS, PROGNOSIS AND MANAGEMENT OF CARDIOVASCULAR DISEASE

(76) Inventors: Nadhipuram V. Bhagavan, Honolulu, HI (US); Chung-eun Ha, Honolulu, HI (US)

Correspondence Address: MINTZ, LEVIN, COHN, FERRIS, GLOVSKY AND POPEO, P.C, ONE FINANCIAL CENTER, BOSTON, MA, 02111, US

(21) Appl. No.: 12/674,765

(22) PCT Filed: Sep. 12, 2008
(86) PCT No.: PCT/US08/76296

§ 371 Date: Feb. 23, 2010

Related U.S. Application Data
(60) Provisional application No. 60/972,168, filed on Sep. 13, 2007.

(51) Int. Cl. C12Q 1/02; C12Q 1/00

(52) U.S. Cl. 435/29; 435/4

(57) ABSTRACT

Methods of detecting myocardial infarction are disclosed based on elevated levels of one or more free fatty acids. The methods may comprise detection of elevated levels of total free fatty acids in a sample relative to average total free fatty acid levels in a control subject without myocardial infarction. Also disclosed are methods to detect myocardial infarction comprising detection of elevated levels of individual free fatty acids in a sample relative to those levels in a control subject and methods comprising determining whether the molar ratio of total free fatty acids to HSA is indicative of myocardial infarction.

### BACKGROUND OF THE INVENTION

1. Field of the Invention

The invention relates to the diagnosis of heart disease by measuring total fatty acid levels or levels of specific individual fatty acids in serum or plasma of a patient. The described methods and compositions are useful for diagnosis as well as for prognosis and management of cardiovascular diseases, such as, for example, cardiac ischemia that leads to myocardial infarction.

2. Description of the Related Art

Ischemic heart disease affects millions of people worldwide, often leading to sudden death by acute myocardial infarction. Cardiac ischemia is often associated with chest pain (angina pectoris), generally caused by atherosclerosis, but asymptomatic individuals can also be at high risk because of hypertension, high serum cholesterol levels or family history. Myocardial ischemia results from the lack of adequate blood flow via the coronary blood vessels to the heart muscle cells, resulting in a deficiency of oxygen and nutrients and eventually compromising their vital functions. Prolonged ischemia can lead to myocardial cell death (necrosis), which is known as myocardial infarction (Ml). During MI, cardiac tissue is damaged, which can result in abnormal cardiac muscle metabolism and contractions.

It is desirable to identify myocardial ischemia before the onset of irreparable myocardial cell damage. Acute myocardial ischemia can result in acute coronary syndromes (ACS), i.e., clinical conditions such as, for example, unstable angina (unpredictable chest pain) and myocardial infarction (myocardial cell death and necrosis; also known as heart attack). In the ideal circumstance, physicians could reliably identify patients with definite ACS and begin appropriate therapy as early as possible, as well as distinguish those without acute coronary ischemia who may be candidates for early discharge without extended observation in the emergency department, chest pain unit, or inpatient wards. However, current diagnostic procedures for heart disease often assess the extent of cardiac tissue damage after symptoms are detected. By then, the disease may have progressed to an extent where AMI is imminent or has occurred. For example, in addition to assessment of the subject's symptoms, acute MI (AMI) or an evolving MI is diagnosed by measuring myocardial proteins in the serum (e.g. creatine kinase MB, troponin I or T) along with electrocardiogram (ECG) studies and imaging procedures. Such measurements necessitate at least 6 hours after symptom onset to exclude MI with high accuracy (Jaffe, A. S., et al. 2000. Circulation 102:1216-1220). ECG and currently available diagnostic blood tests are also often not effective for detecting ischemia because they are designed to monitor infarction-associated tissue damage. Furthermore, ECG monitoring of patients for MI detects the condition in only about half of the patients (Mair J. et al. 1995. Clin. Chem. 41:1266-1272).

Moreover, myocardial ischemic manifestations are vague and multiple. Symptoms are atypical at clinical presentations and may include chest pain (angina), epigastric distress and arm discomfort with exertion or at rest, shortness of breath, nausea, and vomiting. These symptoms may be subtle and are not easily recognized. About one third of patients with acute myocardial infarction do not exhibit chest discomfort during initial clinical presentations. Thus, a sensitive and reliable diagnostic test is needed for diagnosis of cardiac ischemia, especially for high-risk individuals. In particular, identification of a biochemical marker that is sensitive and specific for myocardial ischemia and can be rapidly measured in serum would be clinically valuable.

### SUMMARY OF THE INVENTION

According to one aspect of the invention, total free fatty acid (FFA) levels in a patient can be used to diagnose or monitor myocardial infarction (MI). In some embodiments, the methods comprise: providing a sample from a patient; measuring the total free fatty acid (FFA) level in the sample; and determining whether the total FFA level is indicative of MI.

In some embodiments of the invention, the determining step of the method comprises determining that the total FFA level in the sample is significantly higher than the total FFA level in samples from one or more control subjects that do not have MI. In other embodiments, the determining step comprises determining that the total FFA level in the sample is at least about two-fold greater than the total FFA level in the control subject samples. In some embodiments, the total FFA level is indicative of MI if it is greater than 1 mM. In other embodiments, the total FFA level is indicative of MI if it is greater than 1.3 mM. In still other embodiments, the total FFA level is indicative of MI if it is greater than 1.38 mM.

Embodiments of the invention also include methods of detecting myocardial infarction (MI) comprising: providing a sample from a patient; measuring the total free fatty acid (FFA) level in the sample; measuring the level of HSA in the sample; and determining whether the molar ratio of total FFA:HSA in the sample is indicative of MI.

In some embodiments of the invention, the determining step of the method comprises determining that the molar ratio of total FFA:HSA in the sample is significantly higher than the corresponding ratio in samples from one or more control subjects that do not have MI. In other embodiments, the determining step comprises determining that the molar ratio of total FFA:HSA in the sample is at least about two-fold greater than the corresponding ratio in the control subject samples. In some embodiments, the molar ratio of total FFA:HSA is indicative of MI if it is greater than 1.5. In other embodiments, the molar ratio of total FFA:HSA is indicative of MI if it is greater than 1.7. In still other embodiments, the molar ratio of total FFA:HSA is indicative of MI if it is greater than 1.9.

In some embodiments, the patient is a human.

In some embodiments, the sample is a serum sample. In other embodiments, the sample is a plasma sample. In still other embodiments, the sample is an unclotted whole blood sample.

In another aspect, specific free fatty acid (FFA) levels in a patient can be used to diagnose or monitor myocardial infarction (MI). In embodiments of the invention, methods of detecting myocardial infarction (MI) are provided, comprising: providing a sample from a patient; measuring a level of one or more specific free fatty acids (FFA) in the sample; and determining whether the specific FFA level is greater than control levels found in non-ischemic, non-MI subjects.

In some embodiments of the invention, the specific FFA is selected from one of the following: oleic acid, arachidonic acid, palmitic acid, linoleic acid, stearic acid, palmitoleic acid, eicosapentanoic acid and docosahexanoic acid.

In some embodiments of the invention, the level of two or more of the following specific FFAs are measured: oleic acid, arachidonic acid, palmitic acid, linoleic acid, stearic acid, palmitoleic acid, eicosapentanoic acid and docosahexanoic acid.

In some embodiments, the oleic acid level is indicative of MI if it is greater than 0.39 mM. In some embodiments, the palmitic acid level is indicative of MI if it is greater than 0.2 mM. In some embodiments, the linoleic acid level is indicative of MI if it is greater than 0.21 mM. In some embodiments, the stearic acid level is indicative of MI if it is greater than 0.09 mM. In some embodiments, the palmitoleic acid FFA level is indicative of MI if it is greater than 0.31 mM. In some embodiments, the arachidonic acid FFA level is indicative of MI if it is greater than 0.14 mM.

### BRIEF DESCRIPTION OF THE DRAWINGS

FIGS. 1A and B illustrate measurements of total unesterified fatty acids and HSA-free cobalt activities of human serum albumin (HSA) in patient samples from a control group and an MI group. (A) Molar ratio of total unesterified fatty acid to albumin in serum samples. (B) HSA-free cobalt activities of human serum albumin in serum samples. In both charts, the X-axis represents serum group.

FIGS. 2A-G depict measurements of total and individual free fatty acids detected in myocardial ischemia (MI) and non-myocardial (non-MI) patients. (A) Total unesterified free fatty acid (uFFA) levels. (B) Serum oleic acid (18:1) levels. (C) Serum palmitic acid (16:0) levels. (D) Serum linoleic acid (18:2) levels. (E) Serum stearic acid (18:0) levels. (F) Serum palmitoleic acid (16:1) levels. (G) Serum arachidonic acid (20:4) levels.

### DETAILED DESCRIPTION OF THE PREFERRED EMBODIMENT

Myocardial ischemia causes a compensatory hyperadrenergic state, which is induced by the release of stress-related chemicals (catecholamines and cytokines). The release of such chemicals during myocardial ischemia leads to breakdown of tissue and plasma phospholipids and triacylglycerols of adipose tissues into the bloodstream, which mobilizes and increase plasma concentration of unesterified fatty acids commonly known as free fatty acids (FFAs). Since FFAs are primarily transported bound to human serum albumin (HSA), and since this binding causes structural alterations in HSA, it is contemplated that FFAs contribute to reduced HSA-cobalt [II] binding in the ischemia-modified albumin (IMA) cobalt binding test. The inventors therefore investigated FFAs as useful markers for diagnosis and monitoring of myocardial ischemia.

Throughout the application, although generally discussed in terms of myocardial infarction, embodiments of the invention arc applicable to both myocardial ischemia and myocardial infarction.

Embodiments of the invention relate to methods for detecting myocardial infarction, comprising providing a sample from a patient, measuring the total free fatty acid level in the sample, and determining whether the total free fatty acid level is indicative of myocardial ischemia in the patient. In some embodiments, the sample is a serum sample. In other embodiments, the sample is a plasma sample. In still other embodiments, the sample is an unclotted whole blood sample. In some embodiments, one or more specific free fatty acid levels in the sample can also be measured, as described below.

Embodiments of the invention also include methods for evaluating the risk of myocardial infarction in a subject, comprising providing a sample from a subject, measuring the total free fatty acid level in the sample, and determining whether the total free fatty acid level is indicative of an increased risk for myocardial ischemia in the subject. In some embodiments, the sample is a serum sample. In other embodiments, the sample is a plasma sample. In still other embodiments, the sample is an unclotted whole blood sample.

Embodiments of the invention also relate to methods for detecting myocardial infarction comprising providing a sample from a patient, measuring the level of at least one specific free fatty acid in the sample, and determining whether the level of the at least one free fatty acid is indicative of myocardial ischemia in the patient. In some embodiments, the sample is a serum sample. In other embodiments, the sample is a plasma sample. In still other embodiments, the sample is an unclotted whole blood sample. The at least one specific free fatty acid can be selected from the group of: oleic acid, stearic acid, arachidonic acid, palmitic acid, linoleic acid, eicosapentanoic acid and docosahexanoic acid. In some embodiments the levels of two FFAs are measured. In other embodiments the levels of three, four, five, six or more FFAs arc measured.

Embodiments of the invention also include methods for evaluating the risk of myocardial infarction in a subject, comprising providing a sample from a subject, measuring the level of at least one free fatty acid in the sample, and determining whether the level of the at least one free fatty acid is indicative of myocardial ischemia in the patient. In some embodiments, the sample is a serum sample. In other embodiments, the sample is a plasma sample. In still other embodiments, the sample is an unclotted whole blood sample. The at least one free fatty acid can be selected from the group of: oleic acid, stearic acid, arachidonic acid, palmitic acid, linoleic acid, eicosapentanoic acid and docosahexanoic acid.

In some embodiments of the invention, methods to measure the level of an individual free fatty acid in a sample are provided. In some embodiments, the methods can employ gas liquid chromatography. In other embodiments, the methods can employ HPLC. In additional embodiments, the methods can employ immunological assays. For example, in certain embodiments, the assay can be an immunodiagnostic test for detection of a free fatty acid in a patient sample. In other exemplary embodiments, the assay can be an enzyme-linked immunoassay.

### Assays to Evaluate Myocardial Infarction in Ischemic Patients

The methods and compositions described herein can be used for assessing and evaluating MI in a subject. In earlier studies, a serum-based biochemical test has been found to be useful in the diagnosis of acute myocardial ischemia (Bar-Or, D., et al. 2000. J Emerg Med 19: 311-315). The basic principle of this test involves the N-terminal region of the human serum albumin (HSA) and its inherent affinity for the metal ion cobalt Co(II). HSA is the most abundant multifunctional protein in blood; it consists of 585 amino acid residues (66.5 kDa), is synthesized in the liver, and has a half-life of about 19 days. Serum albumin of myocardial ischemic subjects exhibit reduced binding to cobalt Co(II) compared to serum albumin of non-ischemic subjects. This reduced Co(II) binding to serum albumin was also observed in subjects with transient myocardial ischemia after elective coronary angioplasty surgery (Bar-Or, D., et al. 2001. Am Heart J 141: 985-991).

The inventors confirmed the previous studies (Bar-Or, D. et al. 2000; Bar-Or, D. et al. 2001; Christensen, R. H., et al. 2001. supra) that illustrated that Co(II)—albumin colorimetric assay distinguishes myocardial ischemic patients from non-ischemic patients (P<0.0001) (Bhagavan, N. V., et al. 2003. Clin Chem 49(4)381-585, which is incorporated herein by reference in its entirety). However, it was found that the test is a poor discriminator between ischemic subjects with and without myocardial infarction. The binding of transition metals to the N-terminal region of albumin has been studied. However, the biochemical mechanisms that cause altered Co(II) binding to albumin during ischemia are not well understood, and it appears to be reversible as shown in a study of patients with transient ischemia induced during elective angioplasty surgery (Bar-Or, D., et al. 2001. supra). Co(II)—albumin assay results were abnormal during transient ischemia and returned to baseline values by 6 hours after elective angioplasty. Moreover, in vivo structural modifications that alter Co(II) binding capacity to albumin can potentially occur due to multiple causes such as acidosis, reduced oxygen tension, various ion-pump disruptions, and generation of free radicals.

Consequently, the inventors conducted further research to understand what mechanisms and/or modifications of HSA may potentially occur in vivo to affect Co(II)—albumin interactions (e.g., methylation, N-acetylation, Cu²⁺ mobilization). As HSA is the primary carrier of free fatty acids (FFA) in serum and it can bind up to eleven molecules of FFAs depending on the chain length (Curry, S., et al. 1998. Nat Struct Biol 5:827-835; Bhattacharya, A. A., et al. 2000. J Mol Biol 303:721-732), FFA levels in patient samples have been found to be useful as markers for MI.

### Total Fatty Acid Level Assays

Fatty acids are generally found in more complex molecules through ester or amide bounds. For example, in order to participate in any metabolic process, fatty acids are first activated by being joined in a thioester linkage (R—CO—SCoA) to the sulfhydryl (—SH) group of coenzyme A. The formation of the resulting thioester bond in fatty acyl CoA is a high energy bond that facilitates subsequent metabolic reactions. Free fatty acids, or fatty acids that have not been modified with ester or amide bounds, are bound to serum albumin in order for transport to tissues such as liver, heart and muscle, where they are taken up and oxidized. The isolation of unbound free fatty acid can accordingly be a difficult and laborious process.

As herein described, embodiments of the invention include methods comprising the steps of measuring a total free fatty acid (FFA) level in a sample obtained from a patient; comparing the total FFA level to that of a non-ischemic, non-MI control subject; and determining whether the patient is suffering from myocardial infarction based on the relative difference between the total FFA levels of the patient and the control subject. In additional embodiments, methods are disclosed comprising the steps of: measuring the total free fatty acid level in a sample obtained from a patient; comparing the total level to that of a non-ischemic, non-MI control subject; and determining whether the subject is at risk for myocardial infarction based on the relative difference between the total FFA levels of the patient and the control subject.

In the embodiments described above, certain threshold levels for total free fatty acids are indicative of a diagnosis or of increased risk for MI in a subject. For example, total FFA threshold concentration indicative of a diagnosis or increased risk for MI can be at least about 1 mM, preferably at least about 1.1 mM, more preferably at least about 1.2 mM, 1.3 mM, or 1.4 mM. In some embodiments, the total FFA threshold concentration indicative of a diagnosis or increased risk for MI is at least about 1.38 mM. In additional embodiments, the threshold level can be provided as a molar ratio of total FFA:HSA. For example, a total FFA:HSA ratio indicative of a diagnosis or increased risk for MI can be at least about 1.5, preferably at least about 1,6, 1.7, or 1.8. In some embodiments, a total FFA:HSA ratio indicative of a diagnosis or increased risk for MI is at least about 1.9.

In some embodiments of the invention, total FFA levels can be assayed by any means known in the art. For example, the total FFA levels can be measured by using the WAKO enzymatic colorimetric kit according to the manufacturer's protocol. In other embodiments, total FFA levels can be assayed by developing a calorimetric method to measure FFA converting enzyme.

In embodiments of the invention, HSA levels can be assayed by any means known in the art. For example, HSA levels can be measured by the bromocresol green dye method and by the bicinchoninic acid (BCA) protein assay kit according to the manufacturer's protocol.

### Individual Free Fatty Acid Level Assays

In addition to the measurement of total FFA levels in patient samples, embodiments of the invention include methods comprising the steps of measuring one or more specific free fatty acid (FFA) levels in a sample obtained from a patient; comparing the one or more specific FFA levels to those of non-ischemic, non-MI control subjects; and determining whether the patient is suffering from myocardial infarction based on the relative difference between the individual FFA levels of the patient and the control subject. In some embodiments, the determining step is based on the finding that the one or more specific FFA levels in the patient sample are elevated relative to those in non-ischemic, non-MI control subjects. In additional embodiments, methods are disclosed comprising the steps of: measuring a specific FFA level in a sample obtained from a patient; comparing the specific FFA level to that of non-ischemic, non-MI control subjects; and determining whether the subject is at risk for myocardial infarction based on the relative difference between the specific FFA level of the patient and the control subject. In some embodiments, the determining step is based on the finding that the specific FFA level in the patient sample is elevated relative to those of non-ischemic, non-MI control subjects.

In some embodiments, methods can include comparing the total FFA level and one or more specific FFA levels of a patient to those of a non-ischemic, non-MI control subject; and determining whether the patient is suffering from myocardial infarction based on the relative difference between the total FFA levels and the one or more specific FFA levels of the patient and the control subject. In additional embodiments, methods are disclosed comprising the steps of: measuring the total free fatty acid level and one or more specific FFA levels in a sample obtained from a patient; comparing the total level and specific levels to those of a non-ischemic, non-MI control subject; and determining whether the subject is at risk for myocardial infarction based on the relative difference between the total FFA levels and specific levels of the patient and the control subject.

A variety of specific FFAs are known and can be measured according to various embodiments disclosed herein. For example, the individually measured specific free fatty acids can include, without limitation, oleic acid (oleate), arachidonic acid (arachidonate), palmitic acid (palmitate), linoleic acid (linoleate), stearic acid (stearic acid), palmitoleic acid (palmitate), eicosapentanoic acid (eicosapentaenoate, EPA), docosahcxanoic acid (docosahexaenoate) and others.

In some embodiments, certain threshold levels for specific free fatty acids are indicative of a diagnosis or of increased risk for MI in a subject. In some embodiments, the oleic acid (18:1) threshold concentration indicative of a diagnosis or increased risk for MI can be, for example, at least about 0.39 mM, preferably at least about 0.4, 0.5, 0.6, 0.7 or 0.79 mM. In some embodiments, the oleic acid (18:1) threshold concentration indicative of a diagnosis or increased risk for MI is at least about 0.52 mM.

In some embodiments, the palmitic acid (16:0) threshold concentration indicative of a diagnosis or increased risk for MI can be, for example, at least about 0.2 mM, preferably at least about 0.22, 0.26, 0.3 or 0.39 mM. In some embodiments, the palmitic acid (16:0) threshold concentration indicative of a diagnosis or increased risk for MI is at least about 0.26 mM.

In some embodiments, the linoleic acid (18:2) threshold concentration indicative of a diagnosis or increased risk for MI can be, for example, at least about 0.21 mM, preferably at least about 0.22, 0.25, 0.3 or 0.35 mM. In some embodiments, the linoleic acid (18:2) threshold concentration indicative of a diagnosis or increased risk for MI is at least about 0.22 mM.

In some embodiments, the stearic acid (18:0) threshold concentration indicative of a diagnosis or increased risk for MI can be, for example, at least about 0.09, 0.1, 0.11 or 0.12 mM. In some particular embodiments, the stearic acid (18:0) threshold concentration indicative of a diagnosis or increased risk for MI is at least about 0.09 mM.

In some embodiments, the palmitoleic acid (16:1) threshold concentration indicative of a diagnosis or increased risk for MI can be, for example, at least about 0.031, 0.04 or 0.05 mM. In some particular embodiments, the palmitoleic acid (16:1) threshold concentration indicative of a diagnosis or increased risk for MI is at least about 0.031 mM.

In some embodiments, the arachidonic acid (20:4) threshold concentration indicative of a diagnosis or increased risk for MI can be, for example, at least about 0.014, 0.015, 0.02, 0.025 or 0.03 mM. In some particular embodiments, the arachidonic acid (20:4) threshold concentration indicative of a diagnosis or increased risk for MI is at least about 0.02 mM.

Any method known in the art can be used to measure the level of specific free fatty acids. In some embodiments of the invention, individual FFA levels can be detected by standard chromatography techniques such as gas-liquid chromatography or HPLC using acidic solvent systems. In some embodiments, the sample must be treated prior to analysis by chromatography. For example, the sample can be extracted using solid phase extraction, as described elsewhere (Battistutta, F., et al. 1994. J High Resol Chromatogr 17:662-664, which is incorporated herein by reference in its entirety). In other embodiments, the sample can be treated with solvent extraction (Lalman, J. A., et al. 2004. Journal of the American Oil Chemists' Society 81:105-110, which is incorporated herein by reference in its entirety).

In other embodiments of the invention, individual FFA levels can be detected by an enzyme-linked immunoassay. Monoclonal antibodies against individual FFAs can be developed as known in the art for use in such assays.

### Kits for Evaluation of Myocardial Infarction

Kits are accordingly contemplated for risk evaluation and diagnosis of MI in a patient. In some embodiments, the kit comprises components to measure the total FFA level in a sample. In other embodiments, the kit comprises components to measure the level of at least one specific fatty acid in a sample. In still other embodiments, the kit comprises components to measure the total FFA level and the level of at least one specific fatty acid in a sample.

Fatty Acid Measurement by Immunodiagnostic Assay

In embodiments to determine whether a total or an individual FFA level indicative of a diagnosis for or an increased risk of MI in a patient, threshold levels of total fatty acid or a specific free fatty acid level in a patient sample can be detected. Embodiments to measure the threshold FFA level include an immunodiagnostic assay for detection of a fatty acid analyte in the sample. Exemplary immunodiagnostic assays include, but are not limited to, immunodiagnostic tests, enzyme-linked immunoassays and lateral diffusion assays.

In an exemplary embodiment, the diagnostic assay can be carried out in a well or a 96-well plate for detection of a specific FFA in a sample. For example, a well can be prepared to such that it contains a first binding component that is capable of binding the specific FFA. The first binding component can be, for example, conjugated or cross-linked to the solid surface within the well. A liquid sample from a patient is mixed with a conjugate that is linked to an enzyme, or otherwise labeled for visual detection, that can also bind to free (unbound) binding component in the well. The mixture is applied to the well, and available FFA in the sample competes with the conjugate for binding to the first binding component. Higher concentrations of specific FFA in the sample will reduce the amount of first binding component available to bind the conjugate mixed with the patient sample. Any remaining binding component that does not bind the specific FFA interacts with the conjugate, producing a second complex that either produce a visual cue to indicate the presence of the specific FFA in the sample, or is reacted with a substrate to produce such a visual cue. Alternatively, as described below, the visual cue can signal that the specific FFA is present in the sample above a certain threshold value in the sample. The first binding component can comprise a specific FFA-binding agent, such as, for example, a monoclonal antibody.

To adjust the sensitivity of the diagnostic assay, additional components that compete with the first binding component for binding to the FFA can be employed. The additional components are mixed with the first binding component prior to conjugation or cross-linking of the component to the solid surface in the well in an amount to adjust the sensitivity for a specific FFA, or a group of FFAs. The sensitivity of the test is adjusted so that a positive test result is not given unless a certain threshold of FFA is present in the sample.

The sample used in the test can be any liquid. Preferred samples include, for example, blood, serum, and plasma. The sample is applied to the positive test area and conjugate is subsequently caused to flow from the conjugate source area. The conjugate flows across the membrane and contacts any fatty acid analyte bound to the positive test area, producing a visual signal.

### Examples

The following examples, including the experiments conducted and results achieved are provided for illustrative purposes only and are not to be construed as limiting upon the teachings herein.

### Example 1

### Effect of Total Fatty Acid Levels on Structural Modification of Human Serum Albumin

The concentrations of FFA and HSA were investigated to determine if differences in free fatty acid levels affected Co(II)—albumin interactions in the cobalt binding assay. Total free fatty acid (FFA) and human serum albumin (HSA) concentrations were measured in sera obtained from 33 myocardial ischemic subjects and 54 non-myocardial ischemic subjects. Both ischemic and non-ischemic subjects were of comparable age groups. Total FFA levels were measured by using the WAKO enzymatic colorimetric kit (WAKO Diagnostic Inc.) according to the manufacturer's instructions. HSA levels were measured by the bromocresol green dye method and by the bicinchoninic acid (BCA) protein assay.

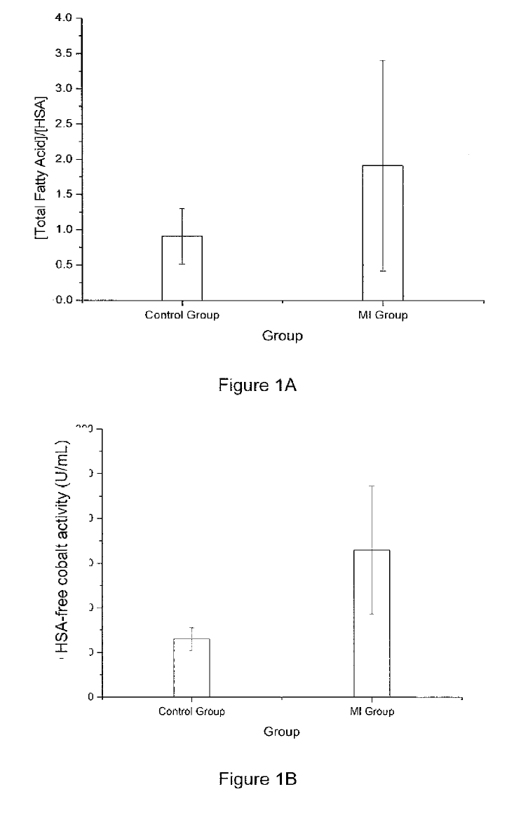

FIG. 1 provides the results of the experiments. FIG. 1A illustrates the molar ratio of total FFA concentration to HSA concentration in sera obtained from control subjects and myocardial infarction (MI) subjects. The molar ratio of total unesterified fatty acid to albumin is depicted for samples from both groups. For the control group, the molar ratio was 0.91±0.39, while the molar ratio was 191±1.49 for the MI group (p<0.001). Mean values are provided with 2 standard deviations. In FIG. 1B, the HSA-free cobalt activities for the same two subject groups, namely “control” and “MI” groups, were 65.06±12.9 U/mL and 164.62±71.74 U/mL, respectively (p<0.001). A greater amount of HSA-free cobalt was detected in the MI group, indicating that reduced Co[II]-HSA binding occurs in the MI patients. Results are expressed as mean values of 54 patients (control group) and 33 patients (MI group), with two standard deviations illustrated by error bars. In summary, the results demonstrate that increased total FFA concentration and decreased cobalt binding activity are observed in the MI group relative to the control group.

### Example 2

### Effect of Individual Fatty Acid Levels on Structural Modification of Human Serum Albumin

The concentrations of twelve unesterified fatty acids were measured in vitro to determine if differences in individual fatty acid levels affect Co(II)—albumin interactions in the cobalt binding assay. The effect of individual fatty acid concentrations on HSA was evaluated in the presence of HSA from two different sources: (1) purified commercial HSA with defined buffer conditions, and (2) USA in pooled normal serum. The cobalt binding assays were performed after incubation of HSA with each unesterified fatty acid for 12 hours at room temperature. The concentrations of the individual FFAs were determined by measuring total FFAs levels before and after the addition of the specific FFAs to pooled normal serum. HSA concentrations were measured by standard colorimetric protein assays. Molar ratios were calculated from the concentration values determined for the individual specific FFAs with respect to the concentrations of HSA measured in each sample.

Table 1 provides the maximum percentage change of cobalt binding activity of commercially purified HSA after incubation with each unesterified fatty acid.

TABLE 1

| Fatty Acid | [Fatty Acid]/[HSA] | Percentage Change (%) |
|---|---|---|
| Laurate | 1.00 | 1.79 (0.81 |
| Myristate | 10.39 | 2.07 (1.14 |
| Palmitic acid | 4.94 | 11.34 ± 0.55 |
| Stearic acid | 1.97 | −0.99 ± 0.85 |
| Arachidate | 0.04 | 0.32 ± 0.83 |
| Behenate | 0.10 | −0.98 ± 1.32 |
| Lignocerate | 0.10 | −2.41 ± 0.72 |
| Palmitoleic acid | 6.06 | 8.06 ± 2.21 |
| Oleic acid | 6.03 | 3.40 ± 1.67 |
| Linoleic acid | 8.21 | 3.64 ± 0.21 |
| Linolenate | 5.58 | 2.54 ± 1.47 |
| Arachidonic acid | 8.18 | 4.70 ± 0.49 |

Table 2 provides the percentage change of cobalt binding activity of HSA in pooled serum after incubation with each unesterified fatty acid.

TABLE 2

| Fatty Acid | [Fatty Acid]/[HSA] | Percentage Change (%) |
|---|---|---|
| Control | 0.83 | 0.27 ± 1.61 |
| Laurate | 7.74 | 5.27 ± 1.74 |
| Myristate | 5.87 | 11.27 ± 0.80 |
| Palmitic acid | 4.18 | 9.80 ± 1.40 |
| Stearic acid | 1.19 | 1.13 ± 0.80 |
| Arachidate | 0.78 | −0.80 ± 2.03 |
| Behenate | 0.77 | −1.07 ± 3.00 |
| Lignocerate | 0.83 | −0.33 ± 1.36 |
| Palmitoleic acid | 8.22 | 12.73 ± 2.33 |
| Oleic acid | 8.34 | 32.33 ± 0.80 |
| Linoleic acid | 7.72 | 10.60 ± 0.72 |
| Linolenate | 4.60 | −14.07 ± 0.64 |
| Arachidonic acid | 8.29 | 37.93 ± 0.46 |

Based on these in vitro results, oleic acid and arachidonic acid (at comparable fatty acid:HSA molar ratios of about 8) have a significant effect on the percent reduction of the Co[II]-HSA binding assay. The effect is most apparent in the assays conducted with HSA in pooled normal serum rather than in those conducted with commercially purified HSA.

The disparate assay results between commercially purified HSA and HSA in pooled serum indicate that the commercial HSA may not be suitable for evaluation of fatty acid levels on Co[II]-HSA binding. Commercial HSA is obtained from pooled serum and can be subjected to both in vivo and in vitro modifications such as glycosylation and truncation at the N-terminus. In previous studies, it has been demonstrated that subjects which express HSA with truncated N-terminal residues will not provide a normal cobalt binding test result (Bhagavan et al. 2003. supra). Thus, further use of commercially purified HSA may not be suitable.

### Example 3

### Determination of Myocardial Infarction in a Patient Based on Total Free Fatty Acid Levels

A human patient presents with severe chest pains. A blood sample is obtained from the patient, and total FFA levels and HSA levels are measured in the sample as described in Example 1. The total FFA concentration, or alternatively, the molar ratio of total FFA:HSA is determined, and the value is compared to baseline levels in non-ischemic, non-MI control subjects. Elevated total FFA values, or alternatively, elevated molar ratios of total FFA:HSA, indicate that the patient is experiencing or has experienced ischemic myocardial infarction.

### Example 4

### Evaluation of Risk for Myocardial Infarction in a Patient Based on Total Free Fatty Acid Levels

A human patient is evaluated in a clinical setting for risk of myocardial infarction. In some embodiments, the patient can be experiencing severe chest pains. In other embodiments, the patient can be undergoing an annual physical examination.

A blood sample is obtained from the patient, and total FFA levels and HSA levels are measured in the sample as described in Example 1. The total FFA concentration, or alternatively, the molar ratio of total FFA:HSA is determined, and the value is compared to baseline levels in non-ischemic, non-MI control subjects. Elevated total FFA values, or alternatively, elevated molar ratios of total FFA:HSA, indicate that the patient is at risk for experiencing ischemic myocardial infarction.

### Example 5

### Determination of Myocardial Infarction in a Patient Based on a Specific Free Fatty Acid Level

A human patient presents with severe chest pains. A blood sample is obtained from the patient, and specific FFA levels and HSA levels are measured in the sample. The specific FFA concentration, or alternatively, the molar ratio of specific FFA:HSA, is determined for one of the following exemplary FFAs: oleic acid, stearic acid, arachidonic acid, palmitic acid, linoleic acid, eicosapentanoic acid, palmitoleic acid and docosahexanoic acid, and the value is compared to baseline levels in non-ischemic, non-MI control subjects. An elevated specific FFA value, or alternatively, an elevated molar ratio of specific FFA:HSA, indicates that the patient is experiencing or has experienced ischemic myocardial infarction.

### Example 6

### Evaluation of Risk for Myocardial Infarction in a Patient Based on a Specific Free Fatty Acid Level

A human patient is evaluated in a clinical setting for risk of myocardial infarction. In some embodiments, the patient can be experiencing severe chest pains. In other embodiments, the patient can be undergoing an annual physical examination.

A blood sample is obtained from the patient, and specific FFA levels and HSA levels are measured in the sample. The specific FFA concentration, or alternatively, the molar ratio of specific FFA:HSA, is determined for one of the following exemplary FFAs: oleic acid, stearic acid, arachidonic acid, palmitic acid, linoleic acid, eicosapentanoic acid, palmitoleic acid and docosahexanoic acid, and the values are compared to baseline levels in non-ischemic, non-MI control subjects. An elevated specific FFA value, or alternatively, an elevated molar ratio of specific FFA:HSA, indicates that the patient is at risk for experiencing ischemic myocardial infarction.

### Example 7

### Determination of Myocardial Infarction in a Patient Based on a Measurement of a Combination of Specific Free Fatty Acid Levels

A human patient presents with severe chest pains. A blood sample is obtained from the patient, and individual specific FFA levels and HSA levels are measured in the sample. The specific FFA concentration, or alternatively, the molar ratio of specific FFA:HSA is determined for at least two of the following exemplary FFAs: oleic acid, stearic acid, arachidonic acid, palmitic acid, linoleic acid, eicosapentanoic acid, palmitoleic acid and docosahexanoic acid, and the value is compared to baseline levels in non-ischemic, non-MI control subjects. The combination of elevated specific FFA values, or alternatively, elevated molar ratios of specific FFA:HSA, indicates that the patient is experiencing or has experienced ischemic myocardial infarction.

### Example 8

### Evaluation of Risk for Myocardial Infarction in a Patient Based on a Measurement of Combinations of Specific Free Fatty Acid Levels

A human patient is evaluated in a clinical setting for risk of myocardial infarction. In some embodiments, the patient can be experiencing severe chest pains. In other embodiments, the patient can be undergoing an annual physical examination.

A blood sample is obtained from the patient, and specific FFA levels and HSA levels are measured in the sample. The specific FFA concentration, or alternatively, the molar ratio of specific FFA:HSA, is determined for at least two of the following exemplary FFAs: oleic acid, stearic acid, arachidonic acid, palmitic acid, linoleic acid, eicosapentanoic acid, palmitoleic acid and docosahexanoic acid, and the value is compared to baseline levels in non-ischemic, non-MI control subjects. The combination of elevated specific FFA values, or alternatively, elevated molar ratios of specific FFA:HSA, indicates that the patient is at risk for suffering from ischemic myocardial infarction.

### Example 9

### Evaluation of Risk for Myocardial Infarction in Patients Based on a Measurement of Total and Individual Free Fatty Acid Levels

The concentrations of total FFA and various individual FFAs were investigated to determine whether the FFA levels are greater than control levels found in non-ischemic, non-MI subjects. Total free fatty acid (FFA) and individual FFA concentrations were measured in sera obtained from 13 myocardial ischemic subjects and 13 non-myocardial ischemic subjects.

Plasma lipids were extracted from serum using the method of Bligh and Dyer (Bligh, E. G. and Dyer, W. J., Can J Biochm Physiol 1959, 37(8):911-917). Mixtures of chloroform and methanol were used to extract the sample in the presence of an odd chain free fatty acid. The chloroform and lipid-containing extract was dried under a stream of nitrogen and reconstituted in a small volume of chloroform_{—} The lipid extract was spotted on a thin layer chromatography plate and developed in a nonpolar solvent system consisting of petroleum ether, diethyl ether and acetic acid (80/20/1) to separate total phospholipids, diglycerides, free cholesterol, free fatty acids, triglycerides and cholesterol esters. The free fatty acid band was isolated from the chromatography plate and derivatized into its corresponding fatty acid methyl ester by the action of sulfuric acid (4%) in excess methanol.

Resulting fatty acid methyl esters were analyzed on a Shimadzu gas chromatograph on a capillary column. The total level of free fatty acid was determined by the comparison of the internal odd chain standard with the total area for the fatty acids eluted.

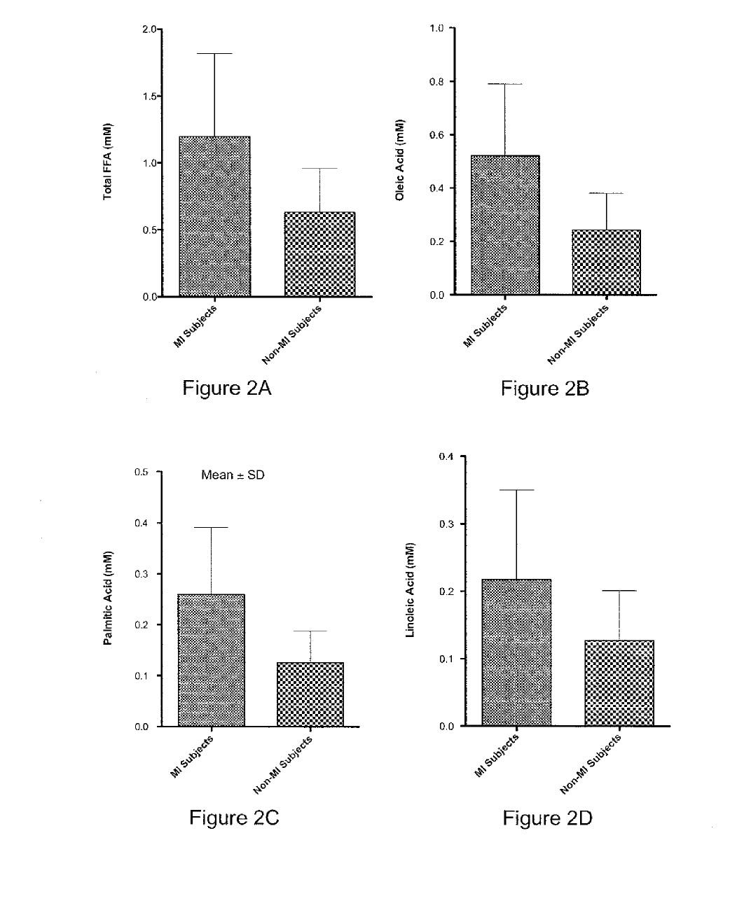

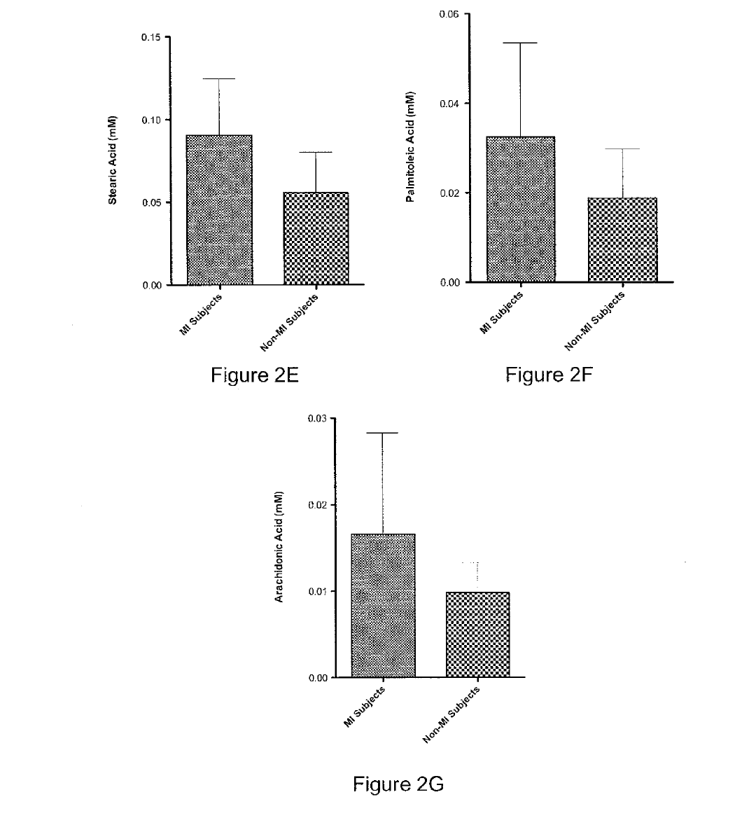

FIG. 2 provides the results of the experiments. FIG. 2A depicts the total unesterified free fatty acid (uFFA) levels of myocardial ischemia (MI) subjects and non-myocardial ischemia (Non-MI) subjects determined by gas chromatography. The graph shows the mean values of serum total uFFA levels obtained from 13 serum samples of MI and Non-MI subject groups with standard deviation (SD). Mean values for Ml subjects and Non-MI subjects were 1.20±0.62 mM and 0.63±0.33 mM, respectively. The students t-test for group comparison was performed and the determined p value (0.0177) was statistically significant (P<0.05). Thus, increased total uFFA concentration is observed in the MI subjects relative to the non-MI subjects.

FIG. 2B depicts serum oleic acid (18:1) levels of myocardial ischemia (MI) subjects and non-myocardial ischemia (Non-MI) subjects. The graph shows the mean values of serum oleic acid levels obtained from 13 serum samples of each sample groups with standard deviation (SD). Mean values for MI subjects and Non-MI subjects were 0.52±0.27 mM and 0.24±0.14 mM, respectively. The students t-test for group comparison was performed and the determined p value (0.003) was statistically significant (P<0.05). Thus, increased oleic acid (18:1) concentration is observed in the MI subjects relative to the non-MI subjects.

FIG. 2C depicts serum palmitic acid (16:0) levels of myocardial ischemia (MI) subjects and non-myocardial ischemia (Non-MI) subjects. The graph shows the mean values of serum palmitic acid levels obtained from 13 serum samples of each sample groups with standard deviation (SD). Mean values for MI subjects and Non-MI subjects were 0.26±0.13 mM and 0.13±0.06 mM, respectively. The students t-test for group comparison was performed and the determined p value (0.0027) was statistically significant (P<0.05). Thus, increased palmitic acid (16:0) concentration is observed in the MI subjects relative to the non-MI subjects.

FIG. 2D depicts serum linoleic acid (18:2) levels of myocardial ischemia (MI) subjects and non-myocardial ischemia (Non-MI) subjects. The graph shows the mean values of serum linoleic acid levels obtained from 13 serum samples of each sample groups with standard deviation (SD). Mean values for MI subjects and Non-MI subjects were 0.22±0.13 mM and 0.13±0.07 mM, respectively. The Students t-test for group comparison was performed and the determined p value (0.0425) was statistically significant (P<0.05). Thus, increased linoleic acid (18:2) concentration is observed in the MI subjects relative to the non-MI subjects.

FIG. 2E depicts serum stearic acid (18:0) levels of myocardial ischemia (MI) subjects and non-myocardial ischemia (Non-MI) subjects. The graph shows the mean values of serum stearic acid levels obtained from 13 serum samples of each sample groups with standard deviation (SD). Mean values for MI subjects and Non-MI subjects were 0.09±0.03 mM and 0.06±0.02 mM, respectively. The students t-test for group comparison was performed and the determined p value (0.007) was statistically significant (P<0.05). Thus, increased stearic acid (18:0) concentration is observed in the MI subjects relative to the non-MI subjects.

FIG. 2F depicts serum palmitoleic acid (16:1) levels of myocardial ischemia (MI) subjects and non-myocardial ischemia (Non-MI) subjects. The graph shows the mean values of serum palmitoleic acid levels obtained from 13 serum samples of each sample groups with standard deviation (SD). Mean values for MI subjects and Non-MI subjects were 0.03±0.02 mM and 0.02±0.01 mM, respectively. The students t-test for group comparison was performed and the determined p value (0.048) was statistically significant (P<0.05). Thus, increased palmitoleic acid (16:1) concentration is observed in the MI subjects relative to the non-MI subjects.

FIG. 2G depicts serum arachidonic acid (20:4) levels of myocardial ischemia (MI) subjects and non-myocardial ischemia (Non-MI) subjects. The graph shows the mean values of serum arachidonic acid levels obtained from 13 serum samples of each sample groups with standard deviation (SD). Mean values for MI subjects and Non-MI subjects were 0.02±0.01 mM and 0.01±0.003 mM, respectively. The students t-test for group comparison was performed and the determined p value (0.058) was statistically not significant (P<0.05). However, increased arachidonic acid (20:4) concentration is observed in the MI subjects relative to the non-MI subjects.

In summary, the results demonstrate that increased total FFA concentration and increased individual FFA concentration are observed in the MI subjects relative to the non-MI subjects.

### INCORPORATION BY REFERENCE

All references cited herein, including patents, patent applications, papers, text books, and the like, and the references cited therein, to the extent that they are not already, are hereby incorporated herein by reference in their entirety.

### Equivalents

The foregoing written specification is considered to be sufficient to enable one skilled in the art to practice the invention. The foregoing description and Examples detail certain preferred embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof.

What is claimed is:

1. A method of detecting myocardial infarction (MI), comprising:
providing a sample from a patient;
measuring the total free fatty acid (FFA) level in the sample; and
determining whether the total FFA level is indicative of MI.

2. The method of claim 1, wherein the patient is a human.

3. The method of claim 1, wherein the sample is a serum sample.

4. The method of claim 1, wherein the sample is a plasma sample.

5. The method of claim 1, wherein the sample is an unclotted whole blood sample.

6. The method of claim 1, wherein the determining step comprises determining that the total FFA level in the sample is significantly higher than the total FFA level in a sample from one or more control subjects that do not have MI.

7. The method of claim 6, wherein the determining step comprises determining that the total FFA level in the sample is at least about two-fold greater than the total FFA level in the control subject sample.

8. The method of claim 1, wherein the total FFA level is indicative of Ml if it is greater than 1 mM.

9. The method of claim 1, wherein the total FFA level is indicative of MI if it is greater than 1.3 mM.

10. The method of claim 1, wherein the total FFA level is indicative of MI if it is greater than 1.38 mM.

11. A method of detecting myocardial infarction (MI), comprising:
providing a sample from a patient;
measuring the total free fatty acid (FFA) level in the sample;
measuring the level of HSA in the sample; and
determining whether the molar ratio of total FFA:HSA in the sample is indicative of MI.

12. The method of claim 11, wherein the patient is a human.

13. The method of claim 11, wherein the sample is a serum sample.

14. The method of claim 11, wherein the sample is a plasma sample.

15. The method of claim 11, wherein the sample is an unclotted whole blood sample.

16. The method of claim 11, wherein the determining step comprises determining that the molar ratio of total FFA:HSA in the sample is significantly higher than the corresponding ratio in a sample from a control subject that does not have MI.

17. The method of claim 16, wherein the determining step comprises determining that the molar ratio of total FFA:HSA in the sample is at least about two-fold greater than the corresponding ratio in the control subject sample.

18. The method of claim 11, wherein the molar ratio of total FFA:HSA is indicative of MI if it is greater than 1.5.

19. The method of claim 11, wherein the molar ratio of total FFA:HSA is indicative of MI if it is greater than 1.7.

20. The method of claim 11, wherein the molar ratio of total FFA:HSA is indicative of MI if it is greater than 1.9.

21. A method of detecting myocardial infarction (MI), comprising:
providing a sample from a patient;
measuring a level of a specific free fatty acid (FFA) in the sample; and
determining whether the specific FFA level is greater than control levels found in non-ischemic, non-MI subjects.

22. The method of claim 21, wherein the specific FFA is selected from one of the following: oleic acid, arachidonic acid, palmitic acid, linoleic acid, stearic acid, palmitoleic acid, eicosapentanoic acid and docosahexanoic acid.

23. The method of claim 21, wherein the level of at least two specific free fatty acids is measured in the sample.

24. The method of claim 23, wherein the at least two specific free fatty acids are selected from the following: oleic acid, arachidonic acid, palmitic acid, linoleic acid, stearic acid, palmitoleic acid, eicosapentanoic acid and docosahexanoic acid.
